(19) United States
(12) Patent Application Publication
(10) Pub. No.: US 2013/0072426 A1
(43) Pub. Date: Mar. 21, 2013

(54) DIAGNOSTIC METHODS FOR OBESITY AND RELATED DISORDERS

(71) Applicant: Giuseppe Matarese, Napoli (IT)

(72) Inventor: Giuseppe Matarese, Napoli (IT)

(73) Assignee: Consiglio Nazionale delle Ricerche, Roma (IT)

(21) Appl. No.: 13/583,233

(22) PCT Filed: Mar. 11, 2011
(86) PCT No.: PCT/EP2011/053673

§ 371 Date: Oct. 9, 2012

Related U.S. Application Data
(60) Provisional application No. 61/312,949, filed on Mar. 11, 2010.

(30) Foreign Application Priority Data
Mar. 11, 2010 (GB) 1004058.2

(51) Int. Cl. G01N 33/566; A61K 31/436; A61P 3/04; G01N 21/64; A61K 38/13

(52) U.S. Cl. 514/4.8; 436/501; 435/7.92; 514/291

(57) ABSTRACT

Methods for detecting the presence of auto-antibodies to antigens expressed by adipose tissue are provided. Also provided are methods for diagnosing, prognosing and treating obesity and/or metabolic syndrome.

### INTRODUCTION

The invention relates to assays that detect the presence of auto-antibodies to antigens expressed by adipose tissue, for example leptin, and the use of their detection in the diagnosis and/or treatment of obesity and/or metabolic syndrome.

### BACKGROUND

Autoimmune diseases and allergic responses are enhanced immune responses to specific antigens which result in pathological conditions (e.g. psoriasis, diabetes, asthma, rheumatoid arthritis, anaphylactic shock, and systemic lupus erythematosus) or discomfort (e.g. allergic rhinitis). These result in a considerable reduction in quality of life and in some situations the response can be life threatening. Inflammation is a complex reaction of the body responding to damage of its cells and vascularised tissues. The inflammatory reaction is phylogenetically and ontogenetically the oldest defense mechanism and both the innate and adaptive immune systems in vertebrates are triggered to destroy the agent(s)/antigens that provoke inflammation. Inflammation can be acute or chronic. An acute inflammatory response is an immediate response by the immune system to a harmful antigen. The response includes vascular dilatation, endothelial and neutrophil cell activation. An acute inflammatory response will either resolve or develop into chronic inflammation. Chronic inflammation is an inflammatory response of prolonged duration, weeks, months, or even indefinitely, whose extended time course is provoked by the persistence of the causative stimulus to inflammation within the tissue. The exact nature, extent and time course of chronic inflammation is variable, and depends on a balance between the causative agent and the attempts of the body to remove it. The mediators of chronic inflammation are both cellular and humoral. Cellular responses include the infiltration of monocytes, macrophages and lymphocytes to the site of inflammation with concomitant tissue damage, angiogenesis and fibrosis. Humoral responses include the production antibodies, for example auto-antibodies and pro-inflammatory cytokines that maintain the inflammatory response.

This disclosure relates to data that is suggestive of obesity being an autoimmune disease wherein auto-antibodies that bind and interfere with the activity of antigens that control diet, food intake and energy metabolism.

At the hypothalamic level, two neuronal populations in the arcuate hypothalamic nucleus (ARC) integrate peripheral signals for nutrition and feeding. One set of neurons expresses the orexigenic (feeding-promoting) neuropeptides agouti-related protein (AgRP) and neuropeptide Y (NPY). These neurons mostly project to other “second order” neurons located in other hypothalamic nuclei such as the paraventricular nuclei (PVH). A second population of ARC neurons expresses the anorexigenic (feeding-inhibiting) products of pro-opiomelanocortin (POMC), the precursor of melanocyte-stimulating hormone (α-MSH) and the cocaine-amphetamine regulated transcript (CART). Hypothalamic neurons respond to peripheral signals such as glucose, leptin, ghrelin, adiponectin, resistin and insulin, by modifying neuropeptide synthesis. When energy intake exceeds expenditure, the expression of orexigenic neuropeptides such as AgRP and NPY decreases. Conversely, the expression of anorexigenic neuropeptides such as CART and POMC increases. Opposite changes occur when energy expenditure exceeds intake. Generally, the two main peripheral signals which control these neurons are leptin, which activates POMC neurons inducing an anorexigenic signal, and ghrelin, which activates AgRP/NPY neurons that induce an orexigenic response. Genetic deficiency in the leptin/LepR system or in the melanocortin system cause early onset obesity, in mice and humans. Only a relatively minor fraction of obese subjects bear one of these heritable mutations. Therefore, other factors such as environment, behaviour and inflammation can help to explain the pathogenesis of most forms of obesity.

This disclosure relates to the observation that obese subjects produce auto-antibodies to antigens produced by adipose tissue, in particular, a high proportion of subjects have auto-antibodies to leptin. This evidence is suggestive that obesity and metabolic syndrome are auto-immune diseases and may benefit from therapeutic intervention to specifically suppress the immune system to delay or prevent obesity and complications associated with obesity. We discovered that in serum of obese subjects it is possible to detect anti-adipose tissue auto-antibodies, and more specifically auto-antibodies against hormones involved in the control of food intake (secreted in high amounts by adipose tissue) such as leptin. We suggest that at least in a consistent fraction of obese and overweight subjects (about 30-50%) there is an autoimmune response against leptin that cause the production of anti-leptin antibodies. These auto-antibodies are produced progressively overtime and induce a decline in the central response to leptin resulting in increase in food intake. Ongoing studies show that auto-antibodies produced can have different activities on leptin in terms of neutralization/stimulation of its activity on the leptin receptor (LepR). Moreover, the presence of these auto-antibodies also determines an inhibition in the passage through the blood-brain-barrier, therefore determining further reduction in hypothalamic responses and central leptin sensitivity.

### STATEMENTS OF INVENTION

According to an aspect of the invention there is provided a diagnostic assay for the detection of auto-antibodies to at least one antigen expressed by adipose cells comprising:
i) providing a sample isolated from a human subject;
ii) contacting the sample with at least one antigen specifically reactive with at least one auto-antibody to form an antigen/auto-antibody complex; and
iii) detecting the complex to determine the presence of auto-antibodies to the antigen[s] as an indicator that the subject is suffering from or susceptible to the development of obesity and/or metabolic syndrome.

“Obesity” is measured in relation to a scale based on Body Mass Index Body Mass Index (BMI) is an approximate measure of obesity, and is calculated by dividing the mass of the individual (in kilograms) by the height squared (in metres squared), i.e. BMI=Weight (kg)/Height² (m²). A healthy BMI range in UK is 18-25, overweight is 25-30 and obese is >30. “Metabolic Syndrome” is a condition that may predispose subjects to type 2 diabetes. The symptoms associated with this syndrome are high blood pressure, dyslipidemia, increased body fat deposition and cardiovascular disease.

In a preferred method of the invention the level of detection of the complex is compared to a control sample of a non-obese or non-metabolic syndrome suffering subject.

In a preferred method of the invention said detection is an ELISA comprising immobilized antigen.

In a preferred method of the invention said assay comprises the following steps:
i) immobilizing an antigen on a solid support;
ii) contacting said immobilized antigen with an isolated sample from a subject comprising antibodies to be tested for specific antigen binding;
iii) incubation of the preparation in ii) for sufficient time to form the antigen/auto-antibody complex;
iv) washing the preparation to remove unbound or non-specifically bound auto-antibody; and
v) contacting the complex with a detection means that detects the complex.

In a preferred method of the invention said detection means is a secondary antibody specifically reactive with the human auto-antibody.

In a preferred method of the invention said secondary antibody binds an isotype selected from then group consisting of: IgA, IgM, IgD, IgE and IgG.

In a preferred method of the invention said secondary antibody binds an isotype selected from the group consisting of: IgG1, IgG2, IgG3 and IgG4.

Antibodies, also known as immunoglobulins, are protein molecules which have specificity for foreign molecules (antigens). Immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

In a preferred method of the invention said solid support is a multi-well microplate

In a preferred embodiment of the invention said auto-antibodies are specifically reactive with one, two, three, four or at least five antigens wherein said antigens are biomarkers expressed by adipose tissue.

In a preferred method of the invention said antigen is a protein or protein produced by adipose tissue.

In a preferred method of the invention said protein ho is selected from the group consisting of: leptin, adiponectin, resistin, apelin, visfatin, omentin, perilipins, vaspin, TNF-alpha.

In a yet further preferred method of the invention said antigen is a protein hormone receptor, for example soluble leptin receptor.

In a preferred method of the invention said antigen is a complex of leptin and soluble leptin receptor.

In a preferred method of the invention said isolated sample is a body fluid sample, for example blood or serum.

The detection of the auto-antibody/antigen complex may be determined by any appropriate means, for example fluorescence or enzyme mediated detection. Labels include fluorochromes, phosphor or laser dye with spectrally isolated absorption or emission characteristics. Suitable fluorochromes include fluorescein, rhodamine, phycoerythrin and Texas Red. Suitable chromogenic dyes include diaminobenzidine. Other labels include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. In the non-limiting examples described below, alkaline phophatas has been employed.

According to a further aspect of the invention there is provided the use of leptin in the diagnosis or prognosis of obesity or metabolic syndrome.

According to an aspect of the invention there is provided a diagnostic assay and a method to treat a subject suffering from or suspected of being susceptible to obesity or metabolic syndrome comprising the steps:
i) providing a sample isolated from a human subject;
ii) contacting the sample with antigen specifically reactive with at least one auto-antibody to form an antigen/auto-antibody complex;
iii) detecting the complex to determine the presence of auto-antibodies to the antigen[s] as an indicator that the subject is susceptible to the development of obesity and/or metabolic syndrome;
iv) determining a treatment regime to prevent and/or control the subject's susceptibility to obesity and/or metabolic disorder; and
v) administering said treatment regime to prevent and/or control obesity and/or metabolic disorder.

In a preferred method of the invention said subject suffers from or is susceptible to obesity.

In an alternative preferred method of the invention said subject suffers from or is susceptible to metabolic disorder.

In a preferred method of the invention the subject is administered an anti-inflammatory drug prior to administration of an anti-insulin resistance drug.

Preferably said anti-inflammatory drug is, for example, rapamycin or cyclosporine.

According to a further aspect of the invention there is provided a kit comprising at least one auto-antigen, means to detect a complex of auto-antigen and auto-antibody and instructions to use said kit components.

In a preferred embodiment of the invention said kit includes one or more secondary antibodies specific for at least one human antibody isotype.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

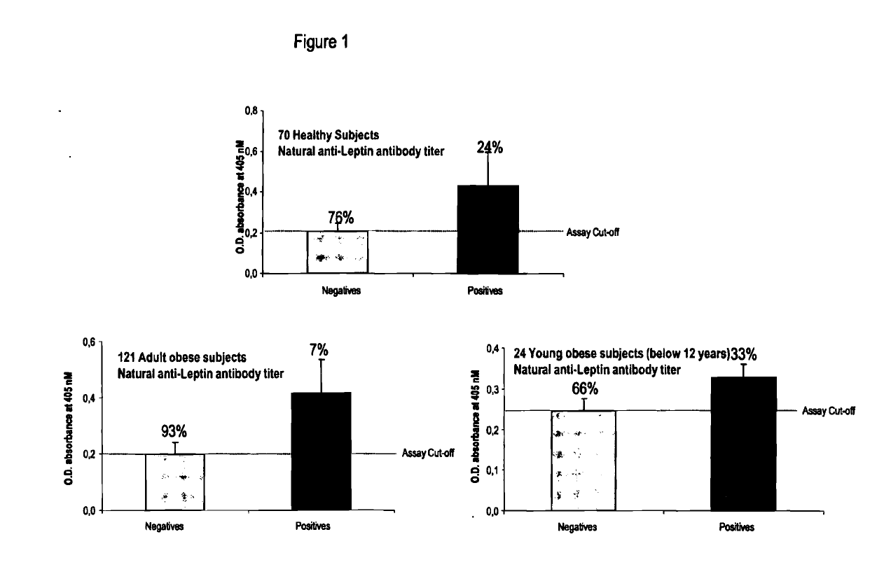

FIG. 1 illustrates an anti-leptin antibody ELISA assay in human and mouse serum. The data presented show that there is a measurable antibody titer in the serum of both healthy and obese subjects. From our analyses in normal non-obese individuals there is about 25% of subjects that show these antibodies; the percentage is lower (7%) in very high obese subjects and is comparable in young obese. In each assay there is a basal binding activity in the serum, that is considered “truly positive” only if it is possible to antagonize the binding of the antibodies upon addition of exogenous recombinant leptin to the assay. The positive are considered only the samples in which the OD values are above the cut-off that is given by the average value of basal reactivity+2 SD of the mean;

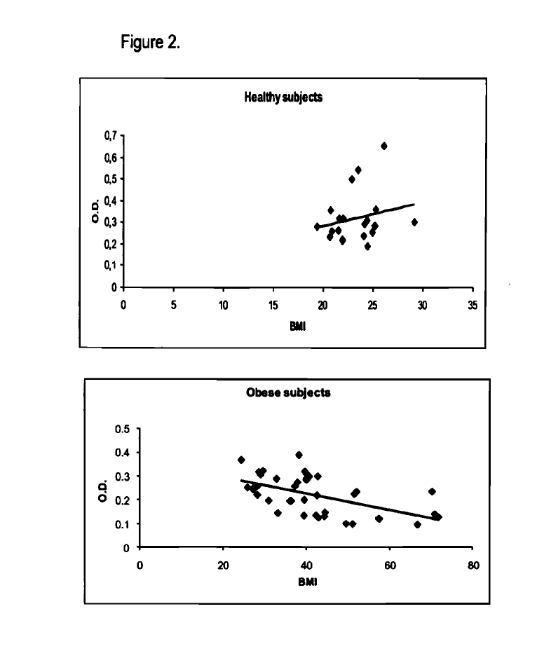

FIG. 2 illustrates a direct correlation between BMI and anti-leptin titer in normal subjects and inverse in obese individuals;

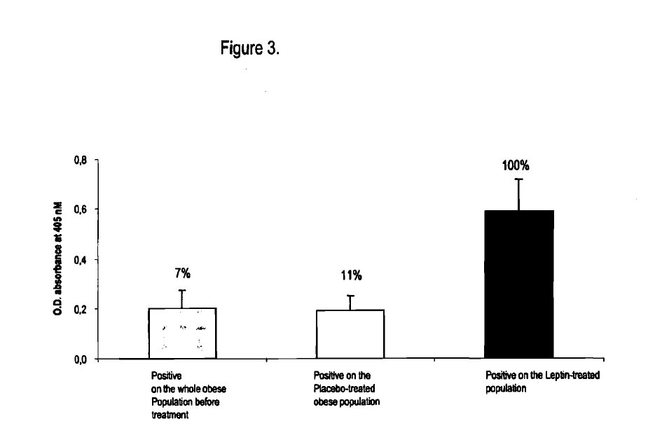

FIG. 3 Illustrates maximum activity and antibody titer has been observed in human obese subjects treated with human met-rec leptin. For at least 12 weeks. In these subjects leptin treatment did not induce significant changes in body weight. The lack of effect by leptin was associated with production of anti-leptin antibodies in 100% of patients; these anti-leptin antibodies were able to bind leptin and inhibit the cross− of leptin into the blood brain barrier. This effect was likely involved in the lack of therapeutic effect of leptin treatment. These antibodies were tested for their capacity to either inhibit or stimulate leptin signalling in a LepR+ cell line transfected with LepR. We analyzed in this cell line proliferation and STAT3 activation upon leptin exposure;

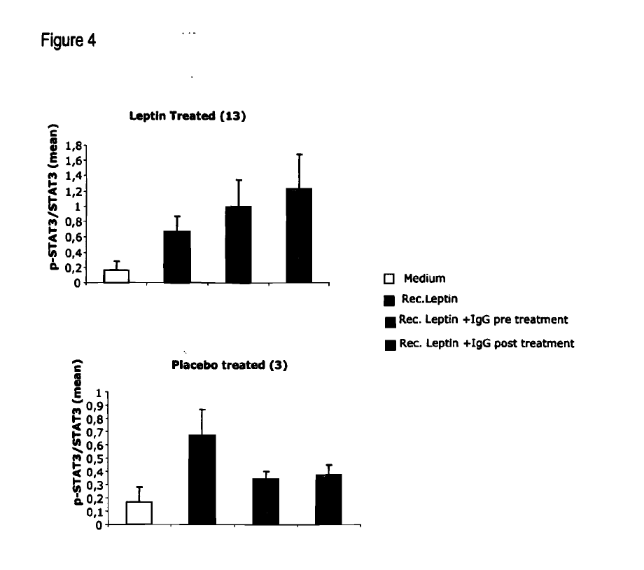

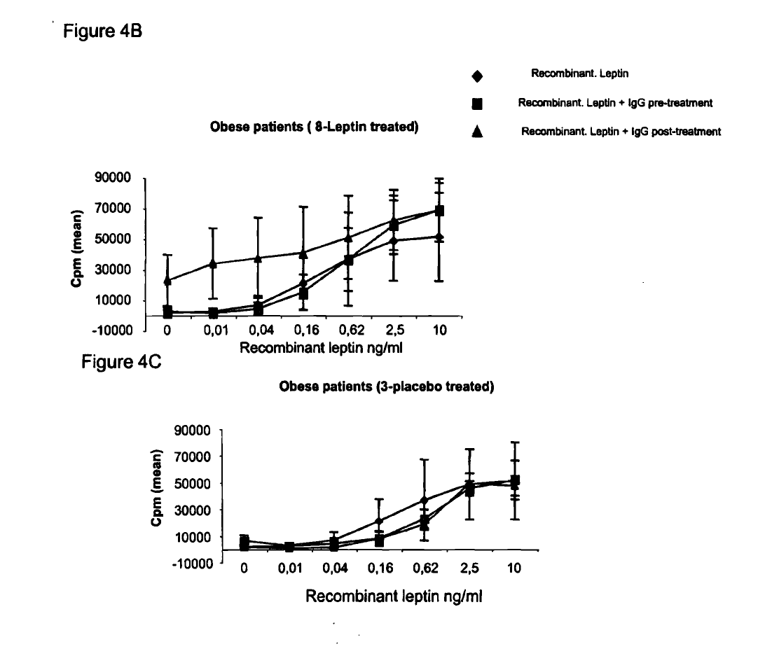

FIGS. 4A, 4B and 4C Anti-leptin antibodies produced during leptin treatment have a stimulatory capacity on a LepR+BAF3 cell line through stimulation of STAT3 activity in leptin treated obese; whereas the natural antibodies have a tendency to inhibit STAT3 signalling and little effect on proliferation;

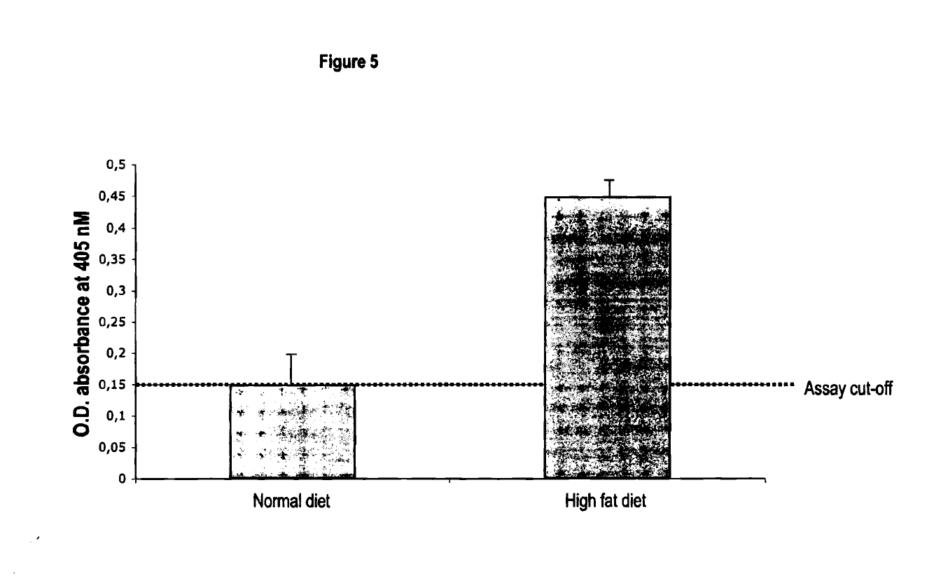

FIG. 5: Mice (15 per group), were administered with normal diet and high fat diet for 4-6 months. Serum was collected and anti-leptin antibodies were measured. We observed an increased natural reactivity as body weight increased. These antibodies correlated with body weight, leptin levels and insulin resistance and reduced tolerance to glucose.

### MATERIALS AND METHODS

### Detection of Anti-Leptin Antibodies and their Functional/Biochemical Activity

Levels of serum anti-leptin antibodies were determined with an in-house developed colorimetric sandwich enzyme-linked immunosorbent assay (ELISA) developed in our laboratory. Briefly, 50 μl of recombinant human leptin (R&D Systems) at final concentration of 10 μg/ml in PBS, pH 7.4 was plate-bound to a 96-well ELISA plate (PBI International SpA). After 16 h at 4° C., the plates were extensively washed with PBS-Tween20 0.05%, blocked with 200 μl of PBS/10% FCS for 2 h, and repeatedly washed. Diluted sera in PBS-0.05% Tween20/10% FCS (from 1/10 to 1/1000) were added at 100 μl/well and incubated for 4 h at room temperature. After five washes, goat anti-human polyvalent immunoglobulins alkaline phosphatase-conjugated Abs (Sigma-Aldrich), were added 100 μ/well for 1 h. The reaction was developed with Sigma-Fast PNPP (p-nitrophenyl phosphate, alkaline phosphatase substrate) (Sigma) and read after 30 min. at 405 in an ELISA plate-reader (Bio-Rad Laboratories, Hercules, Calif.). Standard curves of anti-leptin antibodies were developed in each assay using and anti-leptin monoclonal antibody generated in our laboratory (971212 mAb). Quantification of O.D. values was performed after extrapolation from standard curves of known concentration of anti-leptin antibodies.

The functional activity of anti-leptin antibodies was assessed with the human leptin-receptor (hLepR) transfectant BAF.3 cell line, kindly provided by Prof. Arieh Gertler from The Hebrew University, Rehovot, Israel. Briefly, as hLepR⁺BAF.3 cell proliferation is leptin-dependent, hLepR⁺BAF.3 cells were cultured in flat-bottom 96-well microtiter plates (Becton-Dikinson Falcon) at a density of 5×10³ cells/well in a total volume of 100 μl of RPMI 1640 medium supplemented with 2% FCS (Hyclone-Pierce), 2 mM L-glutamine, 100 U/ml penicillin, 100 μg/ml streptomycin (Life Technologies). Cells were cultured at 37° C. in 100% humidity and 5% CO₂ in the presence of increasing doses of recombinant human leptin (R&D Systems) ranging from 0.01 to 10 ng/ml. Purified IgGs from sera of recombinant leptin, placebo-treated patients and healthy controls were added to cells in all the different conditions at a final concentration of 50 μg/ml. After 48 h [³H]thymidine (0.5 μCi/well) (Perkin-Elmer, Italy) was added to the cultures and cells harvested after 12 h. Radioactivity was measured with a 3-cell-plate scintillation counter (Wallac, Gaithersburg, Md.). As standard of leptin neutralization we utilized our anti-human leptin mAb 971212 at increasing concentrations usually from 0.1 to 25 μg/ml.

We also assessed at biochemical level the capacity of anti-leptin IgGs isolated from leptin or placebo-treated subjects and healthy controls, to affect LepR signalling in BAF.3 cells. Briefly, hLepR⁺BAF.3 cells were incubated for 1 h at 37° C. with recombinant leptin at 2 ng/ml in the presence or absence of IgGs (50 μg/ml) purified from leptin or placebo treated subjects and healthy controls. After 1 h incubation, cell lysates were generated to perform western blotting analyses (see previous section with methods for western blotting) for STAT-3 phosphorylation as readout of LepR signalling.

### EXAMPLE 1

### Presence of Anti-Leptin Immune Response in Normal and Overweight Subjects

In the context of a possible unbalanced anti-adipose tissue autoimmune response as predisposing factor in overweight, insulin resistance and obesity, we have developed in our laboratory an ELISA method to quantify anti-leptin antibodies in the serum. We observed that these antibodies are measurable in both normal and massively obese subjects (FIG. 1). The observed frequency is about 25-30% in normal subjects and 7-12% in adult obese individuals, with a peak up to 30% in early in life obese children (FIG. 1). It is interesting to note that a “basal” reactivity against leptin has been detected in virtually 100% of individuals, thus suggesting that in peripheral blood it is present a “natural” anti-leptin antibody titer that does not necessarily reflect a pathologic condition and that might represent an “idiotypic” autoreactivity necessary to maintain self-tolerance. What is been considered as positive are the antibody titer values higher at least 2 standard deviations of the mean value of basal reactivity and negative controls.

### EXAMPLE 2

### Anti-Leptin Antibodies Correlate with Body Weight and BMI in Normal Individuals but Not in Obese Adults

We measured the correlation between body mass index and body weight in anti-leptin positive subjects and observed a direct correlation between BMI, body weight and anti-leptin antibodies, suggesting that in normal population there is a stratification and a direct correlation between natural anti-leptin antibodies and body weight. In obese adults this correlation was lost whereas in obese children this correlation was maintained and is still under investigation as the number of enrolled subjects is still relatively few. In both cases (adults and children) we are still enrolling subjects to allow a stronger statistics (FIG. 2).

### EXAMPLE 3

### Anti-Leptin Antibodies Correlate with Insulin Resistance and Reduced Glucose Tolerance

On a fraction of these subjects positive to anti-leptin antibodies we analyzed the frequency of insulin resistance and reduced glucose tolerance 8 GTT, and OGTT test). We observed that the presence of anti-leptin antibodies correlated with reduced glucose tolerance. Indeed, both obese and normal subjects positive to antibodies had higher basal glycaemia, and reduced tolerance to glucose upon glucose load (data not shown).

### EXAMPLE 4

### Recombinant Leptin Treatment Induces Production of Anti-Leptin Antibodies that Impair Responsiveness to Leptin Treatment In Vivo

Recombinant leptin treatment has been suggested for treatment of obesity. Leptin treatment was shown to be uneffective to treat obesity. We found in serum of obese subjects during recombinant leptin treatment high titers of anti-leptin antibodies (FIG. 3). These antibodies produced proportionally to leptin administration overtime, and were likely responsible for inefficacy of leptin action as leptin bound to antibodies does not cross the blood brain barrier (BBB) and therefore is unable to affect food intake at hypothalamic level. Surprisingly, these antibodies were shown to be able to have an agonistic activity on proliferation and molecular signalling of a leptin receptor+ (LepR) BAF3 cell line, whose proliferation is dependent on leptin. We measured proliferation of LepR+BAF3 cells in the presence of isolated IgGs from leptin- or placebo-treated subjects (FIGS. 4A, B, C). We observed an agonistic activity and an increased STAT3 phosphorylation, that is known to mediate the intracellular biochemical effects of leptin.

These data suggest that it is possible to elicit, with recombinant leptin treatment, an anti-leptin response and autoreactivity even in non-leptin deficient obese and normal subjects. With this evidence, on one side, we demonstrate that it is possible to induce “artificially” an anti-leptin immunity, and to understand whether it is possible to have a condition of “peripheral leptin resistance” upon leptin treatment, on the other. In addition, despite the antibodies were not neutralizing the effect of leptin in the periphery in terms of signalling, their action hampered significantly the effect of leptin at hypothalamic level as the passage of leptin through the BBB is mediated by the LepR on the choroid plexus. Therefore, despite not neutralizing the antibodies did not allow a powerful leptin effect on food intake in these individuals.

### EXAMPLE 5

### High Fat Diet is Able to Induce an Anti-Leptin Response in Normal Mice, that Correlates with High Body Weight and Reduced Glucose Tolerance

To address whether it is possible to induce an anti-adipose tissue derived leptin immune response by increasing the content of fat in normal mice, we exposed to high fat diet mice for 4-6 months and subsequently quantified the anti-leptin immune response in terms of anti-leptin antibodies. Interestingly, we observed a high anti-leptin antibody titer in these animals (FIG. 5). These data suggest that it is possible to induce a spontaneous anti-leptin response by changing the diet in experimental animals. These data are first to suggest that there may be an autoimmune mediated process against adipose tissue leptin that is induced by high fat diet. Further ongoing studies are in progress to fully characterize immunologically these processes.

1-33. (canceled)

34. A diagnostic method for the detection of auto-antibodies to at least one antigen expressed by adipose cells selected from leptin and a complex of leptin and soluble leptin receptor comprising:
i) providing a sample isolated from a human subject;
ii) contacting the sample with the antigen to form an antigen/auto-antibody complex; and
iii) detecting the complex,
wherein the presence of said complex includes that the subject is suffering from or susceptible to the development of obesity and/or metabolic syndrome.

35. The method of claim 34, wherein the detected complex is compared to a control sample of a non-obese or non-metabolic syndrome-suffering subject.

36. The method of claim 34, wherein said detecting comprises an ELISA which comprises immobilized antigen.

37. The method of claim 34, wherein said assay further comprises the following steps:
i) immobilizing the antigen on a solid support;
ii) contacting said immobilized antigen with an isolated sample from a subject comprising auto-antibodies to be tested for specific antigen binding;
iii) incubation of the preparation in ii) for a sufficient time to form the antigen/auto-antibody complex;
iv) washing the preparation to remove unbound or non-specifically bound auto-antibody; and
v) detecting the complex.

38. The method of claim 34, wherein step iii) comprises detecting the complex with a secondary antibody which specifically reacts with the auto-antibody.

39. The method of claim 38, wherein said secondary antibody binds an antibody isotype selected from the group consisting of: IgA, IgM, IgD, IgE, IgG1, IgG2, IgG3 and IgG4.

40. The method of claim 37, wherein said solid support comprises a multi-well microplate.

41. The method of claim 34, wherein said isolated sample comprises a body fluid.

42. The method of claim 41, wherein said sample comprises blood or serum.

43. A method for the diagnosis or prognosis of obesity or metabolic syndrome, comprising detecting auto-antibodies to at least one antigen expressed by adipose cells selected from leptin and a complex of leptin and soluble leptin receptor from a sample isolated from a human subject.

44. A method for the treatment of obesity or metabolic syndrome comprising administration of an anti-inflammatory drug.

45. The method of claim 44 comprising administration of the anti-inflammatory drug prior to administration of an anti-insulin resistance drug.

46. The method of claim 44, wherein the anti-inflammatory drug is selected from the group consisting of: rapamycin and cyclosporine.

47. A kit for the detection of auto-antibodies to at least one antigen expressed by adipose cells selected from leptin and a complex of leptin and soluble leptin receptor, the kit comprising at least one antigen expressed by adipose cells selected from leptin and a complex of leptin and soluble leptin receptor and instructions for carrying out the method of claim 34.

48. The kit of claim 47, which comprises one or more secondary antibodies specific for at least one human antibody isotype.

49. The kit of claim 47, which comprises means for detecting the complex formed by the at least one antigen and auto-antibodies to said antigen.
